(19) United States
(12) Patent Application Publication
(10) Pub. No.: US 2010/0069910 A1
(43) Pub. Date: Mar. 18, 2010

(54) Osteotomy Guide and Method of Cutting the Medial Distal Tibia Employing the Same

(75) Inventor: Carl T. Hasselman, Oakmont, PA (US)

Correspondence Address: Fay Kaplun & Marcin, LLP, 150 Broadway, suite 702, New York, NY, 10038, US

(73) Assignee: SYNTHES USA, LLC, West Chester, PA (US)

(21) Appl. No.: 12/515,682

(22) PCT Filed: Nov. 15, 2007
(86) PCT No.: PCT/US07/84758

§ 371 Date: May 20, 2009

Related U.S. Application Data
(60) Provisional application No. 60/870,223, filed on Dec. 15, 2006.

(51) Int. Cl. A61B 17/56

(52) U.S. Cl. 606/87

(57) ABSTRACT

An osteotomy guide is provided for cutting a bone, such as the medial distal tibia, to facilitate an ankle replacement. The osteotomy guide includes a positioning device having a number of fixation points, a first guide member structured to align with a first portion of the bone, in order to perform a first procedure, thereon, and a second guide member structured to align with a second portion of the bone, in order to perform a second procedure thereon. The fixation points engage the bone and maintain alignment of the first guide member with respect to the first portion during the first procedure, and maintain alignment of the second guide member with respect to the second portion during the second procedure, in order that the first procedure and the second procedure are substantially reproducible.

### CROSS-REFERENCE TO RELATED APPLICATION

This application claims the benefit of U.S. Provisional Patent Application Ser. No. 60/870,223, filed Dec. 15, 2006.

### BACKGROUND OF THE INVENTION

1. Field of the Invention

The invention relates generally to the ankle joint and, more particularly, to osteotomy guides for cutting a bone of the ankle joint such as, for example, the medial distal tibia. The invention also relates to methods of cutting the medial distal tibia.

2. Background Information

Access to the ankle joint is needed in various instances such as, for example and without limitation, fixing fractures of the talus, repairing cartilage injuries of the ankle joint, and/or in joint replacement.

However, access to the ankle joint is substantially limited by the anatomical structures which surround it. Specifically, the lateral side of the ankle joint is covered by the distal fibula as well as by relatively strong ligaments, and the anterior and posterior sides of the ankle joint are substantially covered by nerves, tendons and blood vessels. Accordingly, access to the ankle joint from these directions is inhibited, and can be dangerous because of the potential for accidental injury to the above structures. Additionally, there are generally well known complications associated with accessing the ankle joint using one of the above approaches. For example, wound healing complications are known to be associated with the anterior approach.

In view of the foregoing, the only other remaining option is to access the ankle joint from the medial side. However, that side of the ankle joint is covered by the medial distal tibia, thus requiring that bone be cut in order to provide the desired access. A procedure for cutting the medial distal tibia has been described in the past. See, for example, U.S. Provisional Patent Application Nos. 60/761,007 entitled “Method of Preparing an Ankle Joint for Replacement,” filed Jan. 20, 2006, and 60/781,634 entitled “Method of Preparing an Ankle Joint for Replacement,” filed Mar. 13, 2006. However, cutting the medial distal tibia to access the ankle joint has traditionally required extreme skill on the part of the surgeon performing the procedure. Among other reasons, this is because the cuts are, in large part, estimated based upon x-rays and clinical experience. As a result, there is significant room for human error in the procedure, and a high degree of variation and inconsistency can result each time the procedure is performed.

There is a need, therefore, for an improved method of cutting the medial distal tibia in a precise manner wherein the method and associated cut can be consistently substantially reproduced.

There is, therefore, room for improvement in methods for cutting of the medial distal tibia.

### SUMMARY OF THE INVENTION

These needs and others are met by embodiments of the invention, which are directed to an osteotomy guide and associated method for cutting a bone such as, for example, the medial distal tibia, for example and without limitation, to facilitate an ankle replacement.

As one aspect of the invention, an osteotomy guide is provided for cutting a bone. The osteotomy guide comprises: a positioning device including a number of fixation points; and a guide member coupled to the positioning device, the guide member being structured to align with a portion of the bone, in order to perform an osteotomy procedure on the bone. The fixation points of the positioning device are structured to engage the bone and maintain alignment of the guide member with respect to the portion of the bone during the osteotomy procedure, in order that the osteotomy procedure is substantially reproducible.

The positioning device may comprise a first elongated pivotal member and a second elongated pivotal member. Each of the first elongated pivotal member and the second elongated pivotal member may include a first end, a second end disposed opposite and distal from the first end, and a pivot point disposed between the first end and the second end. The number of fixation points may be a first fixation point disposed at or about the first end of the first elongated pivotal member, and a second fixation point disposed at or about the first end of the second elongated pivotal member. The pivot point of the first elongated pivotal member may be pivotably coupled to the pivot point of the second elongated pivotal member in order that the first fixation point and the second fixation point are movable toward and away from one another. The first guide member may be coupled to a first portion of the positioning device, and the second guide member may be coupled to a second portion of the positioning device. Alternatively, the positioning device may comprise an elongated member including a first arm, and a second arm coupled to the elongated member, the second arm being movable on the elongated member toward and away from the first arm. The number of fixation points may be a first fixation point disposed on the first arm, and a second fixation point disposed on the second arm. The first guide member may be coupled to a first portion of the positioning device, and wherein the second guide member may be coupled to a second portion of the positioning device.

As another aspect of the invention, an osteotomy guide is provided for cutting a bone. The osteotomy guide comprises: a positioning device including a number of fixation points; a first guide member coupled to the positioning device, the first guide member being structured to align with a first portion of the bone, in order to perform a first procedure on the first portion of the bone; and a second guide member coupled to the positioning device and being structured to align with a second portion of the bone, in order to perform a second procedure on the second portion of the bone. The fixation points of the positioning device are structured to engage the bone and maintain alignment of the first guide member with respect to the first portion of the bone during the first procedure, and to maintain alignment of the second guide member with respect to the second portion of the bone during the second procedure, in order that the first procedure and the second procedure are substantially reproducible.

The positioning device of the osteotomy guide may be a tenaculum, wherein the first fixation point is a first tang of the tenaculum and the second fixation point is a second tang of the tenaculum. Alternatively, the positioning device of the osteotomy guide may be calipers. The calipers may include an actuator, wherein the actuator is operable in a first direction to move the first arm toward the second arm, and in a second direction to move the first arm away from the second arm. The actuator may also include a locking mechanism structured to lock the first arm with respect to the second arm when a desired position of the first arm and the second arm is obtained with respect to the bone.

The first guide member may comprise a housing including a number of conduits. The first procedure may comprise drilling the first portion of the bone with a drill bit, wherein the conduits of the first guide member are structured to receive the drill bit, in order to facilitate the first procedure. The second guide member may comprise at least one guide. Each guide may include a corresponding mounting member and a receiving slot, wherein the corresponding mounting member mounts the guide to the osteotomy guide, and wherein the receiving slot is structured to receive at least a portion of a cutting device. The at least one guide of the guide member may comprise a number of guides and an adapter, wherein the adapter extends outwardly from the osteotomy guide and wherein each of the number of guides includes a receiving slot structured to receive a corresponding cutting tool. Each of the guides may be structured to be removably coupled to the adapter, and may be interchangeable with another one of the number of guides, in order to accommodate a different cutting tool.

As another aspect of the invention, a method comprises: employing an osteotomy guide including a positioning device having a number of fixation points; coupling at least one guide member to the positioning device; engaging the bone with the number of fixation points of the positioning device; aligning the at least one guide member with a first portion of the bone; performing a first procedure on the first portion of the bone; and structuring the number of fixation points of the positioning device to maintain alignment of the at least one guide member with respect to the first portion of the bone during the first procedure, in order that the first procedure is substantially reproducible.

The method may further comprise: providing as the at least one guide member a first guide member and a second guide member; aligning the second guide member with a second portion of the bone; performing a second procedure on the second portion of the bone; and structuring the number of fixation points of the positioning device to maintain alignment of the second guide member with respect to the second portion of the bone during the second procedure, in order that the second procedure is substantially reproducible.

### BRIEF DESCRIPTION OF THE DRAWINGS

A full understanding of the invention can be gained from the following description of the preferred embodiments when read in conjunction with the accompanying drawings in which:

FIG. 1 is an isometric view of an ankle joint and an osteotomy guide for drilling and cutting the medial distal tibia in accordance with an embodiment of the invention;

FIG. 2 is an isometric view of the ankle joint and a portion of the osteotomy guide of FIG. 1, with a first guide member of the osteotomy guide shown just prior to engaging the medial distal tibia;

FIG. 3 is an isometric view of the ankle joint and osteotomy guide of FIG. 2 with the first guide member engaging the medial distal tibia and receiving a drill bit, in accordance with an embodiment of the invention;

FIG. 4 is an isometric view of the ankle joint and osteotomy guide of FIG. 3 after a drilling operation, and rotated to position a second guide member of the osteotomy guide for a cutting operation, in accordance with an embodiment of the invention;

FIG. 5 is an isometric view of the ankle joint and osteotomy guide of FIG. 4, with the second guide member receiving a chisel to perform an osteotomy of the medial malleolus, in accordance with an embodiment of the invention;

FIG. 6 is an isometric view of an osteotomy chisel in accordance with an embodiment of the invention;

FIG. 7 is an isometric view of the ankle joint after completion of an osteotomy, showing the removed portion of the medial malleolus including a chevron-shaped cut and two apertures for use in subsequent repair;

FIGS. 8A and 8B are top plan and side elevation views, respectively, of an osteotomy guide, in accordance with another embodiment of the invention;

FIG. 9 is an exploded end elevation view of a portion of an osteotomy guide and a crescentic-shaped first guide member therefor, in accordance with another embodiment of the invention; and

FIG. 10 is an isometric view of the ankle joint after completion of an osteotomy, showing the removed portion of the medial malleolus including a crescentic-shaped cut and two apertures for use in subsequent repair.

### DESCRIPTION OF THE PREFERRED EMBODIMENTS

For purposes of illustration, embodiments of the invention will be described as applied to an osteotomy guide and associated method for drilling and cutting the medial distal tibia using a drill bit and bone chisel, respectively, although it will become apparent that they could also be applied to facilitate an osteotomy or other cutting or drilling procedure using any known or suitable mechanism or tool (e.g., without limitation, a bit; a saw; a spade; a blade), individually or in any suitable combination.

Directional phrases used herein, such as, for example, front, back, top, bottom, upper, lower, interior, exterior and derivatives thereof, relate to the orientation of the elements shown in the drawings and are not limiting upon the claims unless expressly recited therein.

As employed herein, the term “osteotomy” is employed in accordance with its traditional meaning to refer to the cuffing or otherwise suitable removal or severing of bone.

As employed herein, the terms “cut” and “cutting” refer to the process of slicing, chiseling, or otherwise suitably removing or severing bone.

As employed herein, the terms “drill” and “drilling” refer to the process of creating a hole, bore or aperture in bone.

As employed herein, the term “cutting device” refers to any known or suitable tool or mechanism suitable for “cutting” bone.

As employed herein, the term “drilling device” refers to any known or suitable tool (e.g., without limitation, drill bit) or mechanism for “drilling” bone.

As employed herein, the term “bone” refers to any known or suitable human, animal or artificial structure suitable for use in the body as a component of the skeleton.

As employed herein, the term “guide” refers to any known or suitable mechanism, apparatus or fixture (e.g., without limitation, jig), or suitable combination thereof, for establishing a predetermined desired relationship (e.g., suitably precise alignment) between two or more components in order to perform a procedure (e.g., without limitation, an osteotomy) in a suitably precise and accurate manner, which can be substantially replicated or reproduced.

As employed herein, the terms “fastener” and “fastening mechanism” refer to any known or suitable connecting, securing or tightening material, structure or device and expressly include, but are not limited to, suitable protrusions for securing one component to another, as well as receptacles (e.g., without limitation, recesses; slots; sockets; grooves), combinations of interlocking protrusions and receptacles, welds, and devices such as pins, rivets, screws, bolts and any suitable combination of bolts, nuts (e.g., without limitation, lock nuts) and/or washers.

As employed herein, the statement that two or more parts are “coupled” together shall mean that the parts are joined together either directly or joined through one or more intermediate parts.

As employed herein, the term “number” refers to the quantity one or an integer greater than one (i.e., a plurality).

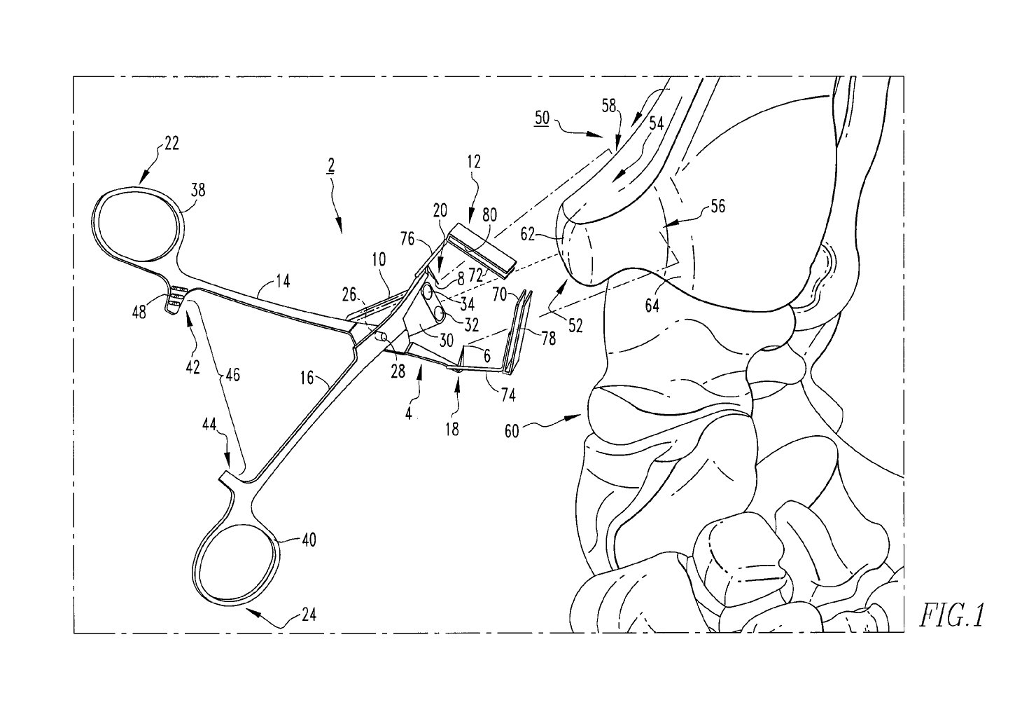

FIGS. 1-5 show an osteotomy guide 2 for reproducibly cutting bone, such as the medial distal tibia 50, and the sequential steps of performing an osteotomy procedure using such osteotomy guide 2, in accordance with embodiments of the invention.

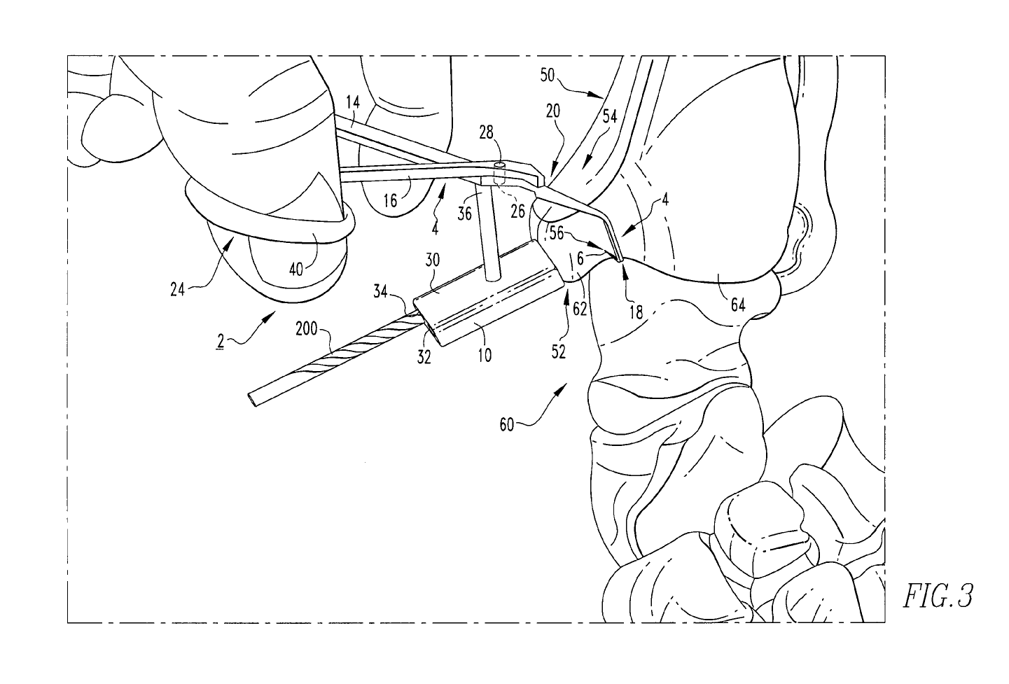

Specifically, as best shown in FIG. 1, the osteotomy guide 2 includes a positioning device 4 having a number of fixation points 6,8. At least one guide member 10,12 is coupled to the positioning device 4 proximate the fixation points 6,8. As will be discussed in greater detail hereinbelow, each guide member 10,12 is structured to align with a corresponding portion of the bone 50, in order to perform the osteotomy procedure thereon. The fixation points such as, for example, the first fixation point 6 and second fixation points 8 shown, are structured to engage the anteromedial corner 56 and posteromedial corner 58, respectively (see FIGS. 3-5), of the medial distal tibia 50 of the ankle joint 60 where the talus 62 meets the medial malleolous 64, when the medial ankle is exposed as shown in FIG. 1.

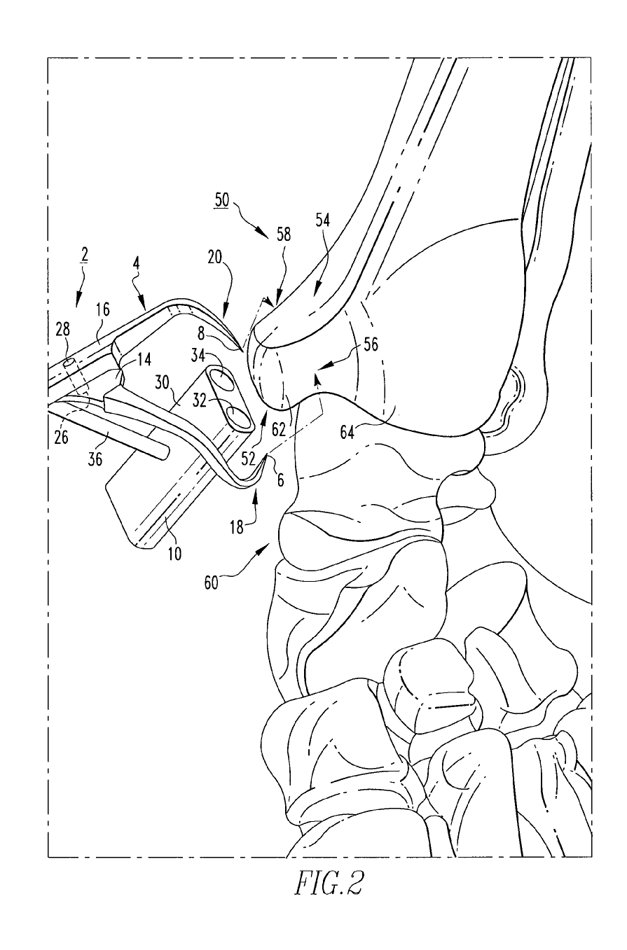

More specifically, the positioning device 4 of the osteotomy guide 2 in the example of FIGS. 1-5 generally comprises a scissor-like instrument such as, for example and without limitation, a tenaculum 4. The first guide member 10 is coupled to a first portion of the tenaculum 4, and the second guide member 12 is coupled to a second portion of the tenaculum 4. The first guide member 10 generally comprises a housing 30 enclosing a pair of conduits 32,34 which are generally parallel with respect to one another, and are structured to receive a drill bit 200, as shown in FIG. 3, or any other suitable drilling device. The example first guide member 10 is coupled to the underside (from the perspective of FIGS. 1-5) of the tenaculum 4 proximate the pivot points 26,28 thereof by way of an elongated shaft 36 (FIGS. 2-5). It will, however, be appreciated that the first guide member 10 could have any known or suitable configuration, including any desirable number of conduits (e.g., 32,34), and could be coupled to the tenaculum 4 using any known or suitable fastening mechanism or configuration other than the elongated shaft 36 shown and described herein, without departing from the scope of the invention.

Continuing to refer to FIG. 1, the example tenaculum 4 includes first and second elongated pivotable members 14,16, each of which has a first end 18,20 and a second end 22,24 disposed opposite and distal from the first end 18,20, respectively. The aforementioned pivot points 26,28 are disposed between the first end 18 and second end 22 of the first elongated pivotable member 14, and the first end 20 and second end 24 of the second elongated pivotable member 16, respectively. The second ends 22,24 include finger holes 38,40, respectively, as well as respective first and second portions 42,44 of a locking mechanism 46, which will be discussed in further detail hereinbelow. The first ends 18,20 of the first and second elongated pivotable members 14,16 include the aforementioned first and second fixation points 6,8, respectively, which, in the example of FIGS. 1-5, comprise relatively sharp tines which are structured to engage the bone 50. The pivot points 26,28 of the first and second elongated pivotable members 14,16 are pivotably coupled together, in order that the first and second fixation points 6,8 are movable toward and away from one another.

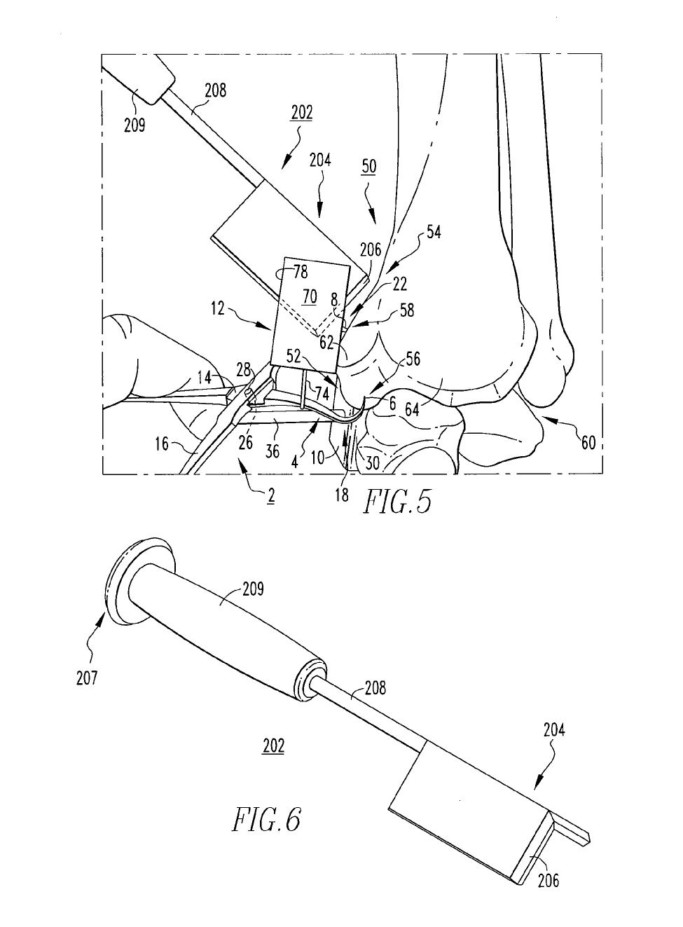

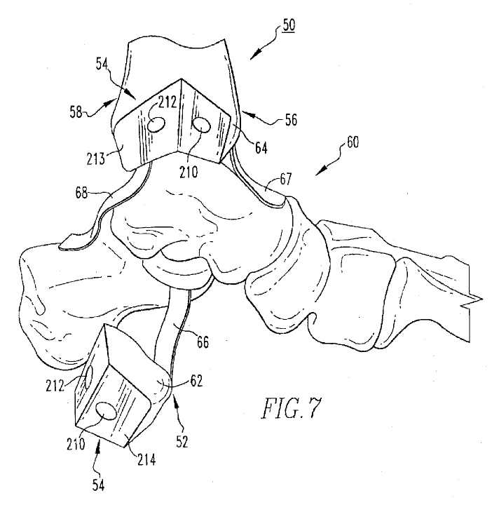

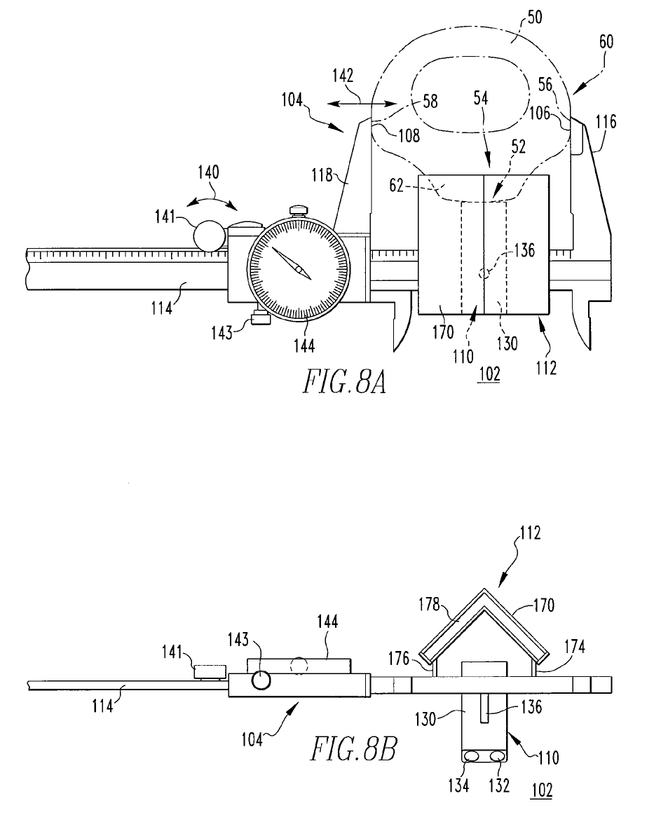

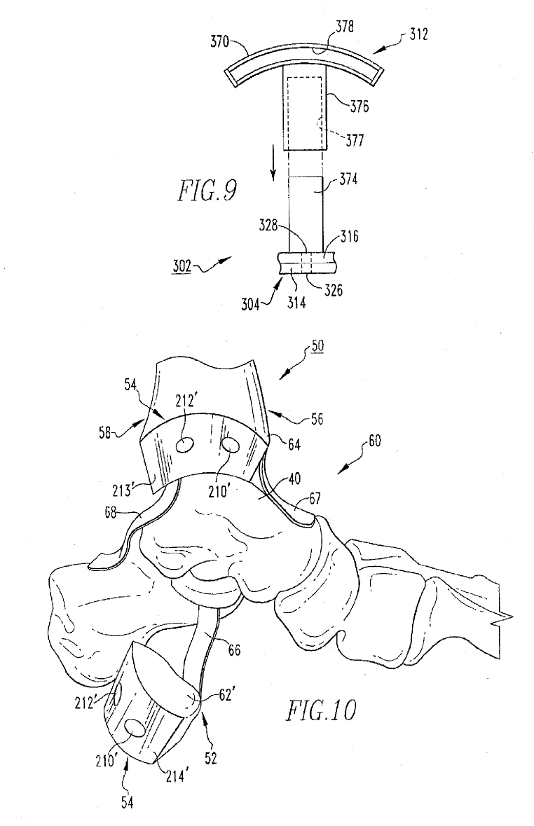

The example second guide member 12 includes a first guide 70 coupled at or about the first end 18 of the first elongated pivotable member 14 by a first guide member 74, and a second guide 72 coupled at or about the first end 20 of the second elongated pivotable member 16 by a suitable second mounting member 76. More specifically, the first and second guides 70,72 of the example second guide member 12 each include a corresponding receiving slot 78,80 structured to receive a suitable cutting device, such as, for example and without limitation, the bone chisel 202 shown in FIGS. 5 and 6. The first and second mounting members 74,76 of the second guide member 12 in the example of FIG. 1 comprise first and second wire members. It will, however, be appreciated that the mounting members 74,76 for the second guide member 12, like the aforementioned shaft 36 for mounting first guide member 10, may comprise any known or suitable mechanism disposed in any suitable number and configuration for fastening the second guide member 12 to the tenaculum 4 or other suitable positioning device (see, for example, caliper 104 of the FIGS. 8A and 8B). It will also be appreciated that although the second guide member 12 in the example of FIG. 1 is shown as comprising two separate guides 70,72, that it could alternatively comprise any known or suitable configuration such as, for example and without limitation, a single guide (see, for example, single guide 170 of second guide member 112 of the osteotomy guide 102 of FIGS. 8A and 8B), without departing from the scope of the invention. It will still further be appreciated that the guide member could have any known or suitable shape (see, for example, crescentic-shaped guide 312 of osteotomy guide 302 of FIG. 9), and that the osteotomy guide could be structured to include an adapter (see, for example, adapter post 374 of FIG. 9) for receiving a wide variety of suitable guides having any desired shape for making a reproducible correspondingly-shaped cut in bone 50.

For simplicity of disclosure and ease of illustration, the second guide member 12 is not shown in FIGS. 2 and 3. In this regard, it will be appreciated that the disclosed osteotomy guides 2 (FIGS. 1-5), 102 (FIGS. 8A and 8B), 302 (FIG. 9) could comprise any suitable number and configuration of first guides (e.g., without limitation, first guide member 10 of FIG. 1) and second guides (e.g., without limitation, second guide member 12 of FIGS. 1-5) individually, or in any suitable combination.

A non-limiting example osteotomy procedure for reproducibly cutting the medical distal tibia 50 of the ankle joint 60, using the disclosed osteotomy guide 2, will now be discussed. Specifically, in a first step which is shown in FIG. 2, the first and second fixation points 6,8 of the osteotomy guide 2 are brought into engagement with the desired interior (e.g., the anteromedial corner 56) and posterior (e.g., posteromedial corner 58) aspects of a medial distal tibia 50 of ankle joint 60. The osteotomy guide 2 will then be locked in the desired position by way of any known or suitable locking mechanism, such as locking mechanism 46, shown in FIG. 1, in order to prevent the osteotomy guide 2 from slipping during subsequent steps of the procedure. The example locking mechanism 46 includes first and second portions 42,44 (FIG. 1) which are structured to overlap and engage one another, in order to lock the tenaculum 4 in the desired engagement with bone 50 (see, for example, FIG. 3). More specifically, the first and second portions 42,44 each include a plurality of ribs 48 (shown only on first portion 42 in FIG. 1), which overlap to engage and interlock with one another (not shown). FIG. 3 shows the osteotomy guide 2 having been locked in the desired position in the foregoing manner.

Next, the osteotomy guide 2 is pivoted downwardly (from the perspective of FIG. 3) or counterclockwise (from the perspective of FIG. 3) until the first guide member 10 is in the desired alignment with the first portion 52 of bone 50, in preparation of the first procedure being performed thereon. The fixation points 6,8 (only fixation point 6 is shown in FIG. 3) help the surgeon (fingers partially shown in FIGS. 3-5) maintain alignment of the first guide member 10 with respect to the first portion 52 of the bone 50 during the first procedure. The first procedure of the example method comprises a drilling procedure wherein two generally parallel holes 210,212 (see FIG. 7; see also holes 210′,212′ of FIG. 10) are drilled into the medial distal tibia 50 starting at the most inferior tip thereof, and extending into the metaphyseal region 54 of the medial distal tibia 50, as shown in FIG. 7. The first guide member 10 facilitates this process, by ensuring that the drilling device, such as the drill bit 200 shown in FIG. 3, is disposed in the proper alignment throughout the procedure. These holes 210,212 (FIG. 7; see also holes 210′,212′ of FIG. 10) will allow for the relatively easy subsequent repair of the cut piece of bone 214 (FIG. 7; see also cut piece of bone 214′ of FIG. 10) once the procedure on the ankle joint 60 has been completed. An osteotomy of the medial distal tibia 50 is described in greater detail, for example, in U.S. Provisional Patent Application Nos. 60/761,007 and 60/781,634 filed Jan. 20, 2006 and Mar. 13, 2006, respectively, which are hereby incorporated herein by reference.

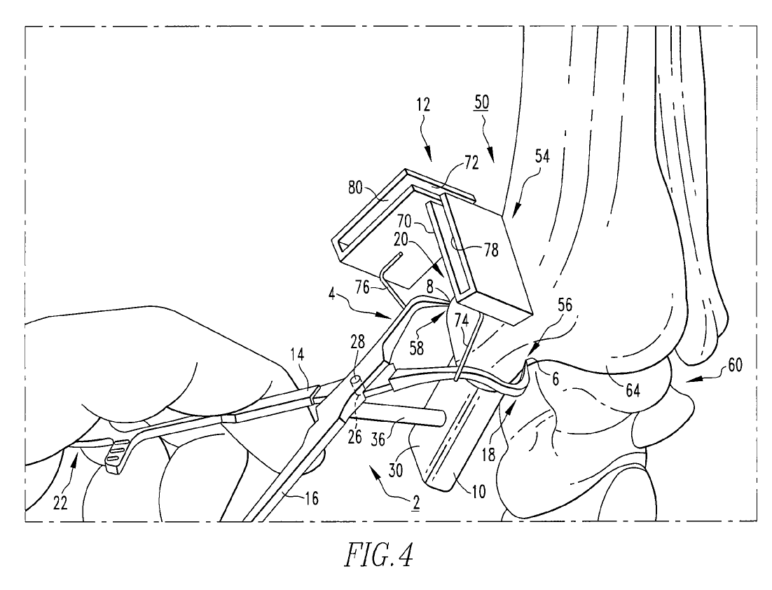

One drill bit 200 is shown within second conduit 34 of the first guide member 10 in the example of FIG. 3. It will be appreciated that the drill bit 200 or other known or suitable drilling device (not shown), may be actuated in any known or suitable manner, (e.g., without limitation, a hand drill (not shown)). Once the first, drilling procedure has been completed, the osteotomy guide 2 is further pivoted downward (from the perspective of FIG. 4) or counterclockwise (from the perspective of FIG. 4) until the second guide member 12 is positioned in the desired aligned position with respect to the second portion 54 of the medial distal tibia 50, shown in FIG. 4. The first and second guides 70,72 of the second guide member 12 are now ready to receive a suitable cutting device such as, for example and without limitation, the bone chisel 202 partially shown in FIG. 5 (see also, FIG. 6). Accordingly, in the next step, which is illustrated in FIG. 5, the second procedure of the example method is performed. Specifically, the bone 50 is cut using the cutting device 202. In this manner, a precise cut of the second portion 54 of the bone 50 can be made to separate a cut piece 214, as shown in FIG. 7. Accordingly, it would be appreciated that the disclosed first and second guide members 10,12 enable an osteotomy procedure wherein drilling and/or cutting procedures may be substantially reproduced, regardless of the skill of the surgeon performing the operation.

FIG. 6 shows the aforementioned bone chisel 202 in greater detail. It will be appreciated, however, that the chisel 202 shown in the example of FIG. 6 is but one non-limiting example of a suitable cutting device for use in accordance with the invention. Specifically, the example bone chisel 202 includes a first end 204, which includes any known or suitable cutting device such as, for example and without limitation, the blade or chisel 206, which is shown. The second end 207 of the bone chisel 202 is structured, for example, to be struck (e.g., hit; tapped) by a suitable tool (e.g., without limitation, a hammer (not shown)). A shaft 208 extends between the first and second ends 204,207, and a hand grip 209 is disposed at or about the second end 207. The example blade or chisel 206 is chevron-shaped or shaped like an inverted capital letter V, in order to make the correspondingly shaped cut in the bone 50, shown in FIG. 7. It will, however, be appreciated that the bone chisel 202, including the blade or chisel 206 thereof could have any known or suitable alternative configuration which is suitable for use with the second guide member 12 of the aforementioned osteotomy guide 2.

FIG. 7 shows the ankle joint 60 and, in particular, the medial distal tibia 50 thereof, after an osteotomy procedure has been performed using the disclosed osteotomy guide 2 (FIGS. 1-5) in accordance with the disclosed method.

Specifically, two generally parallel holes 210,212 have been precisely drilled through the first portion 52 of the medial distal tibia 50 (shown separated in FIG. 7), and a precise chevron-shaped cut 213 has been made in the second portion 54 of the medial distal tibia 50, in order to separate the cut piece 214 of the bone 50, as shown. In this manner, a repair procedure (e.g., without limitation, ankle replacement) may be performed on ankle joint 60, in accordance with any suitable procedure. Following such procedure, the cut piece 214 of the medial distal tibia 50 may be replaced, for example, by inserting screws (not shown) or any other suitable fasteners (not shown) through the holes 210,212 and reconnecting the cut piece 214 to the medial distal tibia 50. As shown, the ankle joint 60 also includes a number of tendons 66,67,68. A more complete description of an ankle replacement procedure is described, for example, in the aforementioned provisional patent applications, have been incorporated herein by reference.

FIGS. 8A and 8B show top plan and side elevation views, respectively, of one non-limiting alternative embodiment of an osteotomy guide 102 in accordance with the invention. Specifically, like the osteotomy guide 2, previously discussed with respect to FIGS. 1-5, the osteotomy guide 102 includes a positioning device 104, first and second fixation points 106,108, which are structured to engage interior and posterior portions 56,58 (shown in simplified form in phantom line drawing in FIG. 8A) of the medial distal tibia 50 (partially shown in simplified form in phantom line drawing in FIG. 8A), and including first and second guide members 110 (shown in hidden line drawing in FIG. 8A) and 112. The fixation points 106,108 of the positioning device which, in the example of FIGS. 8A and 8B comprises calipers 104, are structured to maintain alignment of the first and second guide members 110,112 with respect to first and second portions 52,54 (shown in simplified form in phantom line drawing in FIG. 8A) of bone 50 during first and second procedures which are to be performed respectively thereon, in accordance with the previously disclosed method. In other words, employment of the osteotomy guide 102 to perform an osteotomy procedure is substantially similar to that which was previously discussed hereinabove with respect to osteotomy guide 2 of FIGS. 1-5.

Among the differences of the osteotomy guide 102 with respect to osteotomy guide 2 of FIGS. 1-5, is the employment of the aforementioned calipers 104. Specifically, the calipers 104 comprise an elongated member 114 having a first arm 116, and a second arm 118 which is movably coupled to the elongated member 114. Upon clockwise (from the perspective of FIG. 8A) and counterclockwise (from the perspective of FIG. 8A) movement of an actuator 141 in the direction generally indicated by arrow 140 of FIG. 8A, the second arm 118 is movable toward and away from, respectively, the first arm 116 in the direction generally indicated by arrow 142 of FIG. 8. Once the desired position is achieved, for example, where the first and second fixation points 106,108 engage the bone 50 in the desired locations, as shown in simplified form in phantom line drawing in FIG. 8A, the second arm 118 may be locked in position with respect to the first arm 116, which is generally stationary, by a suitable locking mechanism such as, for example and without limitation, the lock screw 143, which is shown.

As best shown in FIG. 8B, the first guide member 110 of the example osteotomy guide 102 is coupled to a first portion (e.g., without limitation, the bottom side (from the perspective of FIG. 8B)) of the calipers 104 by way of any known or suitable mounting mechanism such as, for example and without limitation, the shaft 136, which is shown. Like the first guide member 10 of osteotomy guide 2, previously discussed in connection with FIGS. 1-5, the first guide member 110 generally comprises a housing 130 enclosing first and second conduits 132,134. The second guide member 112 of the example osteotomy guide 102 is coupled to a second portion (e.g., without limitation, the top side (from the perspective of FIG. 8B)) of the calipers 104 using any known or suitable mounting device such as, for example and without limitation, the first and second mounting members 174,176 shown, which comprise first and second shafts coupled to the elongated member 114 of the calipers 104. Unlike the aforementioned second guide 12, previously discussed with respect to the osteotomy guide 2 of FIGS. 1-5, the second guide member 112 of the osteotomy guide 102 of the example of FIGS. 8A and 8B, comprises one single guide 170 having a single receiving slot 178, which is structured to receive a suitable cutting device (e.g., without limitation, bone chisel 202 of FIGS. 5 and 6) in the manner described hereinabove. It will, however, be appreciated that the second guide member 112 could have any known or suitable alternative shape and/or configuration, without departing from the scope of the invention. For example and without limitation, the second guide member 112 could alternatively comprise first and second guides (not shown) similar to the first and second guides 70,72 of the second guide member 12 of osteotomy guide 2, previously discussed hereinabove with respect to FIGS. 1-5. It will also be appreciated that any known or suitable alternative mounting configuration other than the first and second mounting members 174,176, which are shown, could be employed. For example and without limitation, the guide 170 or guides (not shown) could alternatively be coupled to the first and second arms 116,118 (FIG. 8A) of the calipers 104. Alternatively, without limitation, the mounting members 174,176 could be coupled to the elongated member 114 of the calipers 104 in a suitable movable manner, in order to be adjustable with respect to the elongated member 114. It will still further be appreciated that although the caliper 104 in the example of FIGS. 8A and 8B includes a dial 144 which may be employed, for example and without limitation, to measure the distance between the first and second fixation points 106,108 of the first and second arms 116,118, respectively, that such a dial 144 is not required.

FIGS. 9 and 10 show a non-limiting example alternative embodiment of an osteotomy guide 302, and the medial distal tibia 50 of the ankle joint 60 after undergoing an osteotomy procedure employing the same, respectively. For simplicity of disclosure, only a portion of the osteotomy guide 302 is shown in FIG. 9. It will be appreciated that the remainder of the osteotomy guide 302 and positioning device 304 thereof, may have any known or suitable configuration such as, for example and without limitation, substantially the same configuration as the tenaculum 4, previously discussed in connection with FIGS. 1-5. Specifically, the osteotomy guide 302 of FIG. 9 provides an adaptor post 374 which is suitably coupled to the positioning device 304 at or about the pivot points 326,328 for the first and second elongated pivotal members 314,316 of the device 304, and is structured to receive a wide variety of second guide members 312 in the form of adaptors having any desired shape and configuration for receiving a suitable cutting device (not shown) to precisely and substantially reproducably make the desired correspondingly-shaped cut 213′ (FIG. 10) of the bone 50 (FIG. 10). In the example of FIG. 9, the adaptor 312 has a guide 370 which is arcuate or crescentic-shaped, with a correspondingly shaped slot 378, although it will be appreciated that adaptors (not shown) having any known or suitable alternative shape (not shown) could be employed.

An adaptor member 376, which includes a receptacle 377 (shown in hidden line drawing in FIG. 9) is coupled to the guide 370 and is structured to slide over and engage the adapter post 374. In this manner, a wide variety of different adaptors (e.g., 312) can be interchangeably employed with the osteotomy guide. In the example of FIG. 9, the adaptor member 376 and receptacle 377 thereof are generally square in shape, in order to receive the adapter post 374 of the osteotomy guide 302, which is also generally square. Such generally square configuration prevents one component (e.g., the adaptor guide 312) from undesirably moving (e.g., rotating) with respect to another component (e.g., the positioning device 304) of the osteotomy guide 302. It will, however be appreciated that any suitable alternative configuration and/or fastening mechanism could be employed to accomplish this goal.

The aforementioned adaptor guide 312 can be employed individually, or in combination with a first guide member (not shown), which is substantially similar to first guide members 10, previously discussed with respect to FIGS. 1-5, and 110, previously discussed with respect to FIGS. 8A and 8B. Accordingly, the method disclosed hereinabove can be employed using the osteotomy guide 302 of FIG. 9 to produce the end result shown in FIG. 10. Specifically, holes 210′,212′ can be precisely and substantially reproducibly drilled, and a crescentic-shaped cut 213′ can be precisely and substantially reproducibly made to provide the cut piece 214′ of the talus 62′, shown. It will be appreciate that although it is possible that the cuts 213 and 213′ shown in FIGS. 7 and 10, respectively, may be generally known, that the osteotomy guide 2 (FIGS. 1-5),102 (FIGS. 8A and 8B),302 (FIG. 9) and corresponding method disclosed herein to substantially reproducibly make such cuts 213, 213′ and others (not shown), are believed to be novel in accordance with the embodiments disclosed herein.

Accordingly, it will be appreciated that the disclosed osteotomy guides 2,102,302 and method of cutting the medial distal tibia 50 employing the same, enable a variety of different osteotomy procedures to be performed on the ankle joint 60 in a precise manner which is substantially reproducible, regardless of the level of skill of the surgeon performing the operation.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

1-22. (canceled)

23. An osteotomy guide for cutting a bone, the osteotomy guide comprising:
a positioning device including a number of fixation points; and
a guide member coupled to the positioning device, the guide member being structured to align with a portion of the bone in order to perform an osteotomy procedure on the bone,
wherein the number of fixation points are structured to engage the bone and maintain alignment of the guide member with respect to the portion of the bone during the osteotomy procedure so that the osteotomy procedure is substantially reproducible.

24. The osteotomy guide of claim 23, wherein the positioning device comprises a first elongated pivotal member and a second elongated pivotal member, wherein each of the first and second elongated pivotal members includes a first end, a second end disposed opposite and distal from the first end, and a pivot point disposed between the first end and the second end; wherein the number of fixation points includes a first fixation point disposed at or about the first end of the first elongated pivotal member, and a second fixation point disposed at or about the first end of the second elongated pivotal member; wherein the pivot point of the first elongated pivotal member is pivotably coupled to the pivot point of the second elongated pivotal member in order that the first and second fixation points are movable toward and away from one another and wherein the first guide member is coupled to a first portion of the positioning device and the second guide member is coupled to a second portion of the positioning device.

25. The osteotomy guide of claim 23, wherein the positioning device comprises an elongated member including a first arm and a second arm, the second arm being movable toward and away from the first arm, wherein the number of fixation points comprises a first fixation point disposed on the first arm, and a second fixation point disposed on the second arm; wherein the first guide member is coupled to a first portion of the positioning device; and wherein the second guide member is coupled to a second portion of the positioning device.

26. The osteotomy guide of claim 23, wherein the bone is the medial distal tibia of an ankle joint; wherein the ankle joint includes an anteromedial corner and a posteromedial corner; wherein the number of fixation points are a first fixation point and a second fixation point; wherein the first fixation point is structured to engage the anteromedial corner; and wherein the second fixation point is structured to engage the posteromedial corner.

27. An osteotomy guide for cutting a bone, the osteotomy guide comprising:
a positioning device including a number of fixation points;
a first guide member coupled to the positioning device, the first guide member being structured to align with a first portion of the bone in order to perform a first procedure on the first portion of the bone; and
a second guide member coupled to the positioning device and being structured to align with a second portion of the bone in order to perform a second procedure on the second portion of the bone,
wherein the number of fixation points are structured to engage the bone and maintain alignment of the first guide member with respect to the first portion of the bone during the first procedure, and to maintain alignment of the second guide member with respect to the second portion of the bone during the second procedure, in order that the first procedure and the second procedure are substantially reproducible.

28. The osteotomy guide of claim 27, wherein the positioning device comprises a first elongated pivotal member and a second elongated pivotal member, each of the first and second elongated pivotal members including a first end, a second end disposed opposite and distal from the first end, and a pivot point disposed between the first end and the second end; wherein the number of fixation points includes a first fixation point disposed at or about the first end of the first elongated pivotal member, and a second fixation point disposed at or about the first end of the second elongated pivotal member; wherein the pivot point of the first elongated pivotal member is pivotably coupled to the pivot point of the second elongated pivotal member in order that the first and second fixation points are movable toward and away from one another; and wherein the first guide member is coupled to a first portion of the positioning device and the second guide member is coupled to a second portion of the positioning device.

29. The osteotomy guide of claim 28, wherein the positioning device is a tenaculum; wherein the first fixation point is a first tang of the tenaculum; and wherein the second fixation point is a second tang of the tenaculum, wherein the tenaculum comprises a pair of finger holes and a locking mechanism structured to maintain the position of the first tang and the second tang of the tenaculum with respect to the bone.

30. The osteotomy guide of claim 27, wherein the positioning device comprises an elongated member including a first arm and a second, the second arm being movable toward and away from the first arm; wherein the number of fixation points includes a first fixation point disposed on the first arm and a second fixation point disposed on the second arm; and wherein the first guide member is coupled to a first portion of the positioning device and the second guide member is coupled to a second portion of the positioning device.

31. The osteotomy guide of claim 30, wherein the positioning device is calipers, the calipers including an actuator; wherein the actuator is operable in a first direction to move the first arm toward the second arm, and in a second direction to move the first arm away from the second arm; and wherein the actuator includes a locking mechanism structured to lock the first arm with respect to the second arm when a desired positions of the first and second arms are obtained with respect to the bone.

32. The osteotomy guide of claim 27, wherein the first guide member comprises a housing including a number of conduits; wherein the first procedure comprises drilling the first portion of the bone with a drill bit; and wherein the number of conduits of the first guide member are structured to receive the drill bit in order to facilitate the first procedure.

33. The osteotomy guide of claim 32, wherein the first guide member further comprises an elongated post including a first end and a second end disposed opposite and distal from the first end; wherein the first end of the elongated post is coupled to the positioning device; and wherein the housing of the first guide member is coupled to the second end of the elongated post.

34. The osteotomy guide of claim 27, wherein the second guide member comprises at least one guide; wherein the at least one guide includes a corresponding mounting member and a receiving slot, the corresponding mounting member mounting the at least one guide to the osteotomy guide, the receiving slot being structured to receive at least a portion of a cutting device.

35. The osteotomy guide of claim 34, wherein the at least one guide comprises a first guide coupled to the first elongated pivotal member by a first mounting member, and a second guide coupled to the second elongated pivotal member by a second mounting member; wherein the first guide includes a first receiving slot; wherein the second guide includes a second receiving slot; and wherein the first guide and the second guide cooperate to receiving the cutting device in the first and second receiving slots.

36. The osteotomy guide of claim 34, wherein the at least one guide comprises a number of guides and an adapter, the adapter extending outwardly from the osteotomy guide; wherein each of the number of guides includes a receiving slot structured to receive a corresponding cutting tool; wherein each of the number of guides is structured to be removably coupled to the adapter; and wherein each of the number of guides is interchangeable with another one of the number of guides in order to accommodate a different cutting tool.

37. The osteotomy guide of claim 27, wherein the bone is the medial distal tibia of an ankle joint; wherein the ankle joint includes an anteromedial corner and a posteromedial corner; wherein the number of fixation points are a first fixation point and a second fixation point; wherein the first fixation point is structured to engage the anteromedial corner; and wherein the second fixation point is structured to engage the posteromedial corner.

38. A method comprising:
employing an osteotomy guide including a positioning device having a number of fixation points;
coupling at least one guide member to the positioning device;
engaging the bone with the number of fixation points of the positioning device;
aligning the at least one guide member with a first portion of the bone;
performing a first procedure on the first portion of the bone; and
structuring the number of fixation points of the positioning device to maintain alignment of the at least one guide member with respect to the first portion of the bone during the first procedure, in order that the first procedure is substantially reproducible.

39. The method of claim 38, further comprising:
engaging a predetermined interior aspect of a medial distal tibia of an ankle joint with the first fixation point;
engaging a predetermined posterior aspect of the medial distal tibia of the ankle joint with the second fixation point; and
locking the osteotomy guide in a desired position with respect to the medial distal tibia.

40. The method of claim 38, further comprising:
inserting a drill bit through a first hole of a housing of a first guide member;
drilling a first hole in the first portion of the bone;
inserting the drill bit through a second hole of the housing of the guide member; and
drilling a second hole in the first portion of the bone, the second hole being parallel to the first hole.

41. The method of claim 38, further comprising:
providing as the at least one guide member a first guide member and a second guide member;
aligning the second guide member with a second portion of the bone;
performing a second procedure on the second portion of the bone; and
structuring the number of fixation points of the positioning device to maintain alignment of the second guide member with respect to the second portion of the bone during the second procedure, in order that the second procedure is substantially reproducible.

42. The method of claim 41, further comprising:
inserting a cutting tool through a corresponding at least one receiving slot of the second guide member;
cutting the second portion of the bone; and
separating a cut piece of the second portion of the bone.

43. The method of claim 42, further comprising:
reconnecting the cut piece of the second bone in its original orientation; and
performing an ankle replacement.
